(19) United States
(12) Patent Application Publication
(10) Pub. No.: US 2007/0173660 A1
(43) Pub. Date: Jul. 26, 2007

(54) Catalytic composition and process for asymmetric hydrogenation

(76) Inventors: Lee David Proctor, Denbighshire (GB); Anthony John Warr, Chester (GB); Elliot James Lathom, South Wirral (GB)

Correspondence Address: BROOKS KUSHMAN P.C., 1000 TOWN CENTER, TWENTY-SECOND FLOOR, SOUTHFIELD, MI, 48075, US

(21) Appl. No.: 10/557,749

(22) PCT Filed: Apr. 26, 2004
(86) PCT No.: PCT/GB04/01755

§ 371 Date: Oct. 30, 2006

(30) Foreign Application Priority Data
May 21, 2003 (GB) 0311658.9

(51) Int. Cl. B01J 31/00; C07C 69/66

(52) U.S. Cl. 560/179; 502/150; 502/162

(57) ABSTRACT

The present invention concerns a catalytic composition comprising a catalyst effective for catalysing asymmetric hydrogenation reactions, which catalyst requires acid activation, an acidic material effective for activating the catalyst, and a buffering compound or composition capable of forming, in the presence of the acidic material, an acetal, a ketal, a hemiacetal, and/or a hemiketal. The invention also relates to an asymmetric hydrogenation process utilising such a catalytic composition, and to the use of such a catalytic composition for improving the enantiomeric excess of a desired asymmetrically hydrogenated product.

The present invention relates to a process for asymmetric hydrogenation catalysis, more particularly to such a process performed using an acid-activated hydrogenation catalyst, and to a catalytic composition for use in such a process.

Asymmetric hydrogenation reactions are used in a wide variety of chemical processes, in particular in the manufacture of pharmaceutical intermediates. One particularly significant commercial area at present is in the manufacture of so-called statin drugs, which are used to reduce cholesterol and/or triglyceride levels in the body. Examples of current statin drugs include Atorvastatin (Lipitor™), Fluvastatin (Lescol™) and Rosuvastatin (Crestor™).

WO-A-98/04543 discloses a one pot process for the preparation and isolation of esters of (S)-3,4-O-isopropylidine-3,4-dihydroxybutanoic acid, cyclic othoesters of (S)-3,4-dihydroxybutanoic acid, and (S)-3-hydroxybutyrolactone from a carbohydrate substrate.

U.S. Pat. No. 5,292,939 discloses a process for the preparation of 3,4-dihydroxybutanoic acid from a glucose source.

Useful pharmaceutical intermediates can be formed by the enantioselective hydrogenation of ,βketoesters. The hydrogenation is catalyzed by halogen-containing BINAP-Ru(II) complexes (Tetrahedron Letters, Vol. 32, No. 33, pp 4163-4166, 1991). The BINAP ligand (2,2′-bis (diphenylphosphino)-1,1′-binaphthyl) has the formula (1):

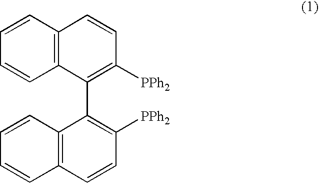

U.S. Pat. No. 6,162,951 discloses processes for the preparation of BINAP catalysts suitable for use in catalyzing asymmetric hydrogenation reactions. The use of Ru(OCOCH₃)₂[{S}-BINAP] in the enantioselective hydrogenation of ethyl 4-chloroacetoacetate is reported by Kitamura et al in Tetrahedron Letters, Vol. 29, No. 13, pp 1555-1556, 1988. Kitamura et al report that the reaction (scherne A) proceeds within 5 minutes giving the (R)-alcohol in 97% in enantiomeric excess. The same reaction was investigated by Pavlov et al in Russian Chemical Bulletin, Vol. 49, No. 4, April, 2000, pp 728-731. Pavlov et al studied the effects of the nature of the solvent, the reaction temperature, the pressure, addition of acids, and the reagent ratio on the yield and degree of an enantiomeric enrichment of the reaction products.

A substantial report in connection with reductions of 1,3-dicarbonyl systems with ruthenium-biarylbisphosphine catalysts has been prepared by Ager and Laneman, reported in Tetrahedron, Asymmetry, Vol. 8, No. 20, pp 3327-3355, 1997 .

EP-A-0295109 teaches a process for preparing an optically active alcohol which comprises a symmetrically hydrogenating a β-keto acid derivative in the presence of a ruthenium-optically active phosphine complex as a catalyst. The resulting alcohol is said to have a high optical purity. Other examples of asymmetric hydrogenation reactions, and catalysts therefor, are disclosed in U.S. Pat. Nos. 5,198,561, 4,739,085, 4,962,242, 5,198,562, 4,691,037, 4,954,644 and 4,994,590.

Our co-pending UK application No. 0211716.6 discloses a continuous process for the enantioselective catalytic hydrogenation of β-ketoesters. Our co-pending UK application No. 0211715.8 discloses a continuous process for cyanation of the resulting hydrogenated material.

One of the problems associated with asymmetric hydrogenation reactions in general, and with asymmetric hydrogenation of β-ketoesters in particular, is to maximise the enantiomeric excess of the desired asymmetrically hydrogenated product over its unwanted enantiomer. It is an object of the present invention to provide such an improvement.

According to the present invention there is provided a catalytic composition comprising a catalyst effective for catalysing asymmetric hydrogenation reactions, which catalyst requires acid activation, an acidic material effective for activating the catalyst, and a buffering compound or composition capable of forming, in the presence of the acidic material, an acetal, a ketal, a hemiacetal, and/or a hemiketal.

Many catalysts which are effective for enantioselective hydrogenation require acid activation. Such catalysts include BINAP or other bisaryl bisphosphine-based ligand catalysts, for example [NH₂Et₂]⁺[RuCl{p-MeO-BINAP}₂{μ-Cl}₃]⁻, [NH₂Et₂]⁺RuCl(p-MeO-BINAP)₂(μ-Cl)₃], [RuI(p-cymene)(p-MeO-BINAP)], [RuI(p-cymene)(p-Tol-BINAP)]I, [RuI(p-cymene)(m-Tol-BINAP)]I, [RuI(p-cymene)(3,5-(t-Bu)₂-BINAP)]I, [RuI(p-cymene)(p-Cl-BINAP)]I, [RuI(p-cymene)(p-F-BINAP)]I, [RuI(p-cymene)(3,5-(Me)₂-BINAP)]I, [RuI(p-cymene)(H₈-BINAP)]I, [RuI(p-cymene)(BIMOP)]I, [RuI(p-cymene)(FUMOP)]I, [RuI(p-cymene)(BIFUP)]I, [RuI(p-cymene)(BIPHEM)]I, [RuI(p-cymene)(MeOl-BIPHEP)]I, [RuCl₂(tetraMe-BITIANP)(DMF)ₙ], [RuCl₂(BITIANP)(DMF)ₙ], [RuBr₂(BIPHEMP)], [RuBr₂(MeO-BIPHEMP)], [RuCl₂(BINAP)]₂(MeCN), [RuCl₂(p-TolBINAP)]₂(MeCN), [RuCl₂(MeO-BIPHEP)]₂(MeCN), [RuCl₂(BIPHEP)]₂(MeCN), [RuCl₂(BIPHEMP)]₂, or [Ru(³-2-Me-allyl)₂(MeO-BIPHEP)] or a combination of two or more thereof.

However, acidic conditions in asymmetric hydrogenation tend to lower the enantiomeric excess of the desired product. A possible mechanistic explanation for this is provided with reference to the following Figures, in which:

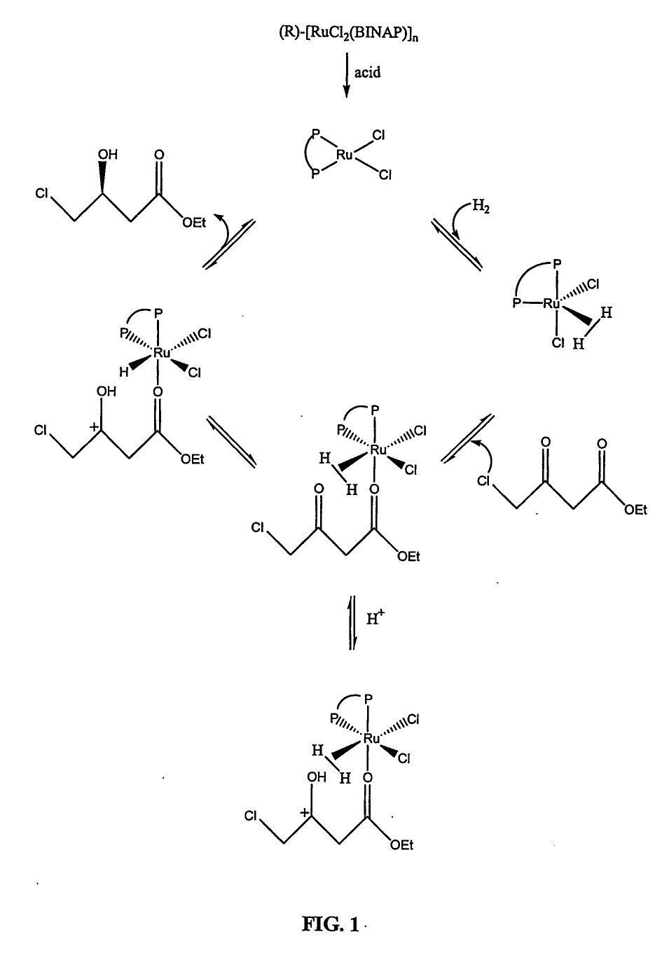

FIG. 1 shows a possible mechanism for the asymmetric hydrogenation of ethyl-4-chloroacetoacetate in the presence of a BINAP catalyst;

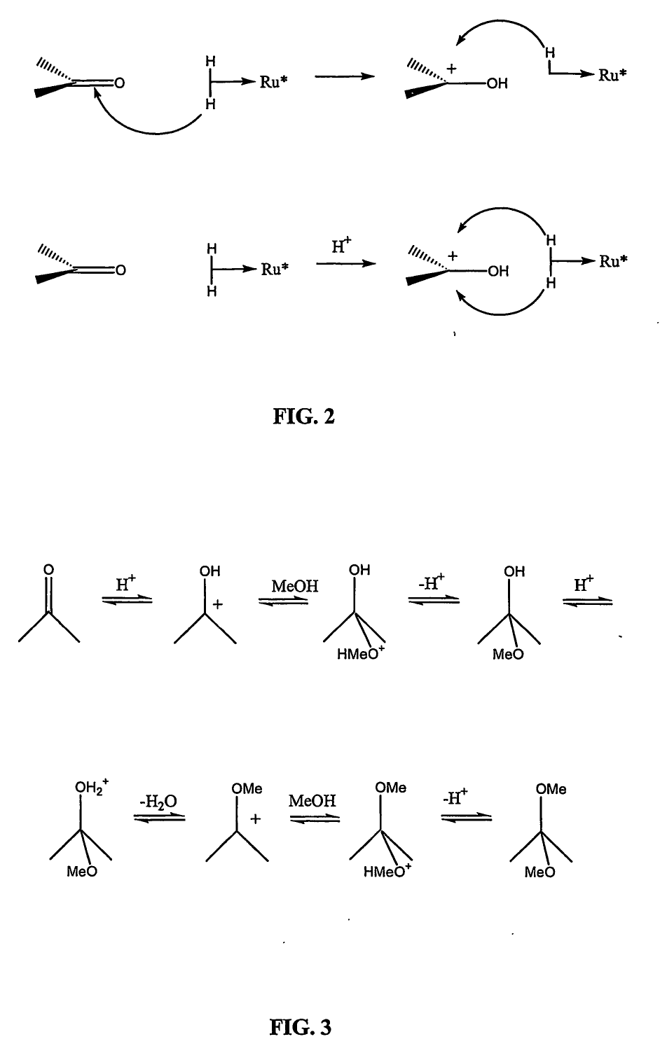

FIG. 2 shows in more detail the enantiomerically crucial hydrogenation step in FIG. 1; and

FIG. 3 provides a possible mechanistic explanation of the buffering activity of an acetone/methanol mixture.

Referring to FIGS. 1, it will be seen that the β-keto group on the substrate is hydrogenated sequentially, the first hydrogenation step being effected by a hydrogen atom coordinated with the BINAP catalyst or, because an acid equilibrium is established, by a hydrogen ion from the acid solution. As is shown clearly in FIG. 2, the origin of the first hydrogenation has an important impact on enantioselectivity. If the first hydrogenation is effected by coordinated hydrogen, the enantiomeric excess is high because there remains only one coordinated hydrogen to effect the second hydrogenation. If the first hydrogenation is effected by hydrogen ions in the acid solution, the enantiomeric excess is low because there remain two coordinated hydrogens which can then attack from either side, giving different enantiomers as a result.

The enantiomeric excess of the desired product may be significantly improved by incorporating a buffering compound or composition in the reaction mixture. This may have the effect of driving the aforesaid equilibrium (shown in FIG. 1) such that the first hydrogenation is effected by coordinated hydrogen, in preference to hydrogen ions from the acid solution.

Also provided in accordance with the invention is a process for the enantioselective catalytic hydrogenation of a hydrogenatable substrate comprising contacting the substrate with hydrogen and with a catalyst effective for enantioselective hydrogenation of the substrate, which catalyst requires acid activation, in the presence of an acidic material and a buffering compound or composition capable of forming, in the presence of the acidic material, an acetal, a ketal, a hemiacetal, and/or a hemiketal, under conditions effective for enantioselective hydrogenation of the substrate.

Buffering compounds and compositions for use in accordance with the invention suitably comprise mixtures of one or more aldehydes and/or ketones with one or more alcohols. Examples include one or more of forrnaldehyde, acetaldehyde, propionaldehyde, n-butyraldehyde, benzaldehyde, p-tolualdehyde, salicyclaldehyde, phenylacetaldehyde, α-methylvaleraldehyde, β-methylvaleraldehyde, isocaproaldehyde, acetone, methyl ethyl ketone, methyl n-propyl ketone, ethyl ketone, methyl isopropyl ketone, benzyl methyl ketone, acetophenone, n-butyrophenone and propylalcohol, isopropylalcohol, n-butylalcohol, isobutylalcohol, sec-butyl alcohol and tert-butylalcohol, but other compositions will be apparent to those skilled in the art. One particularly preferred buffering composition is acetone/methanol.

Referring to FIG. 3, there is shown a possible mechanistic explanation for the buffering activity of an acetone/methanol mixture. It is thought (although the scope of the invention is not to be considered as limited by such explanation) that the buffering action of the mixture allows sufficient hydrogen ions in solution to activate the hydrogenation catalyst but, in “mopping up” excess hydrogen ions, drives the equilibrium shown in FIG. 1 in favour of the enantioselective hydrogenation route (ie away from the intermediate depicted at the bottom of FIG. 1).

The process of the invention may suitably be operated as a batch or continuous process. The reaction temperature is preferably maintained at least about 75° C., more preferably at least about 90° C. and even more preferably at least about 100° C. In one preferred process according to the invention, the reaction temperature is from about 100 to about 150° C.

The buffering compound or composition suitable for use in the invention may act as a solvent for the hydrogenatable substrate.

In one preferred process according to the invention there is provided a continuous process for the enantioselective catalytic hydrogenation of β-ketoesters comprising:
(a) providing a catalytic hydrogenation zone maintained under conditions of temperature and pressure effective for the catalytic hydrogenation of β-ketoesters;
(b) continuously supplying to the catalytic hydrogenation zone a substrate comprising a β-ketoester to be hydrogenated, a catalyst, requiring acid activation, effective for enantioselective hydrogenation of the β-ketoester, an acidic material effective for activation of the catalyst, a buffering compound or composition capable of forming, in the presence of the acidic material, an acetal, a ketal, a hemiacetal, and/or a hemiketal and hydrogen;
(c) contacting the substrate, the catalyst and the hydrogen in the hydrogenation zone for a residence time effective for at least partial enantioselective catalytic hydrogenation of the β-ketoester;
(d) continuously withdrawing from the hydrogenation zone a reaction product mixture comprising enantioselectively hydrogenated β-ketoester, unreacted β-ketoester, catalyst and hydrogen;
(e) supplying the reaction product mixture to a separation zone and separating at least some of the enantioselectively hydrogenated β-ketoester from the reaction product mixture;
(f) withdrawing the separated enantioselectively hydrogenated β-ketoester as product; and
(g) optionally supplying at least part of the remaining material from the separation zone to the hydrogenation zone.

The β-ketoester is preferably ethyl-4-chloroacetoacetate but is suitably of the formula (2): 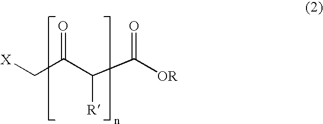
wherein X, R and R′ are independently selected from hydrogen, optionally substituted alkyl, aryl, aryl alkyl or alkaryl groups or optionally substituted cyclo alkyl groups; and wherein X may alternatively be selected from fluorine, chlorine, bromine, iodine, mesylates, tosylates, sulphonate esters, tetra alcyl ammonium and other suitable leaving groups; and n is from 1 to 4.

The β-ketoester may have from 1 to 4 keto groups and may, for example, be a β, δ-diketoester.

Preferably, the hydrogenation zone is maintained at a pressure of at least about 75 bar, more preferably at least about 90 bar and still more preferably at least about 100 bar. In one preferred process according to the invention, the hydrogenation zone is maintained under conditions of from about 100 to about 150 bar.

A key requirement in the manufacture of a symmetrically hydrogenated substrates in general, and β-ketoesters in particular, is the so-called “enantiomeric excess” in the product of the desired enantiomer over the non-desired enantiomer. In the process of the invention, the enantiomeric excess in the product is preferably greater than about 95%, more preferably greater than about 96%, yet more preferably greater than about 97% and most preferably greater than about 98%, for example about 99% or more.

Also provided in accordance with the present invention is a use of a buffering compound or composition in a process for the asymmetric catalytic hydrogenation of a substrate in the presence of an effective catalyst requiring acid activation, and of an acidic material for effecting such activation, which buffering compound or composition has the capacity to form an acetal, a ketal, a hemiacetal, and/or a hemiketal in the presence of the acidic material, to improve the enantiomeric excess of desired asymmetrically hydrogenated product.

The invention will now be more particularly described with reference to the following Examples.

### EXAMPLE 1 (COMPARATIVE)

A 600 ml stainless steel Parr reactor was charged with ethanol (340 ml) and ethyl-4-chloroacetoacetate (53 g). The reactor agitator was started and the speed set to 600 rpm. The reactor was pressurised using nitrogen to 7 bar and stirring continued for 5 minutes. After 5 minutes the reactor was slowly vented to ambient pressure, the pressurisation/depressurisation cycle was repeated for a total of five times to ensure complete removal of dissolved oxygen. At the end of the last cycle the reactor set-point temperature was adjusted to 95° C. (R)-[RuCl₂(BINAP)]n catalyst was accurately weighed (23 mg) into a catalyst transfer vessel and the vessel then purged using nitrogen for 5 minutes. The catalyst was flushed from the transfer vessel using deoxygenated solvent into a 100 ml stainless steel injection bomb which was attached to the Parr reactor. When the Parr reactor temperature was between 95° C. and 100° C. the injection bomb was pressurised to 100 bar using hydrogen. Appropriate valves were then opened to transfer the catalyst mixture and hydrogen into the reactor. The contents of the reactor were stirred at 600 rpm for 30 minutes before being cooled to less than 30° C. The reactor was then slowly vented to ambient pressure. The reactor contents were transferred into a 1 L rotary film evaporator flask and the mixture evaporated to constant weight by application of vacuum and by using a heated water bath. The residue was subjected to pot to pot distillation under vacuum to afford a clear colourless oily liquid product of ethyl (S)-(−)-4-chloro-3-hydroxybutyrate in >98% yield, >98% purity and 94% enantiomeric excess.

### EXAMPLE 2

A 600 ml stainless steel Parr reactor was charged with ethanol (170 ml), acetone (170 ml) and ethyl-4-chloroacetoacetate (53 g). The reactor agitator was started and the speed set to 600 rpm. The reactor was pressurised using nitrogen to 7 bar and stirring continued for 5 minutes. After 5 minutes the reactor was slowly vented to ambient pressure, the pressurisation/depressurisation cycle was repeated for a total of five times to ensure complete removal of dissolved oxygen. At the end of the last cycle the reactor set-point temperature was adjusted to 95° C. (R)-[RuCl₂(BINAP)]n catalyst was accurately weighed (23 mg) into a catalyst transfer vessel and the vessel then purged using nitrogen for 5 minutes. The catalyst was flushed from the transfer vessel using deoxygenated solvent into a 100 ml stainless steel injection bomb which was attached to the Parr reactor. When the Parr reactor temperature was between 95° C. and 100° C. the injection bomb was pressurised to 100 bar using hydrogen. Appropriate valves were then opened to transfer the catalyst mixture and hydrogen into the reactor. The contents of the reactor were stirred at 600 rpm for 30 minutes before being cooled to less than 30° C. The reactor was then slowly vented to ambient pressure. The reactor contents were transferred into a 1 L rotary film evaporator flask and the mixture evaporated to constant weight by application of vacuum and by using a heated water bath. The residue was subjected to pot to pot distillation under vacuum to afford a clear colourless oily liquid product of ethyl (S)-(−)-4-chloro-3-hydroxybutyrate in >98% yield, >99% purity and >98% enantiomeric excess.

### EXAMPLE 3 (COMPARATIVE)

A 600 ml stainless steel Parr reactor was charged with ethanol (340 ml) and 6-chloro-3,5-dioxo-hexanoic acid tert-butyl ester (76 g). The reactor agitator was started and the speed set to 600 rpm. The reactor was pressurised using nitrogen to 7 bar and stirring continued for 5 minutes. After 5 minutes the reactor was slowly vented to ambient pressure, the pressurisation/depressurisation cycle was repeated for a total of five times to ensure complete removal of dissolved oxygen. At the end of the last cycle the reactor set-point temperature was adjusted to 95° C. (R)-[RuCl₂(BINAP)]n catalyst was accurately weighed (23 mg) into a catalyst transfer vessel and the vessel then purged using nitrogen for 5 minutes. The catalyst was flushed from the transfer vessel using deoxygenated solvent into a 100 ml stainless steel injection bomb which was attached to the Parr reactor. When the Parr reactor temperature was between 95° C. and 100° C. the injection bomb was pressurised to 100 bar using hydrogen. Appropriate valves were then opened to transfer the catalyst mixture and hydrogen into the reactor. The contents of the reactor were stirred at 600 rpm for 30 minutes before being cooled to less than 30° C. The reactor was then slowly vented to ambient pressure. The reactor contents were transferred into a 1 L rotary film evaporator flask and the mixture evaporated to constant weight by application of vacuum and by using a heated water bath. The residue was subjected to pot to pot distillation under vacuum to afford a clear colourless oily liquid product of 3R,5S-(6-chloromethyl-2,2-dimethyl-[1,3]dioxin-4-yl)-acetic acid tert-butyl ester in >90% yield, >88% purity and 92% enantiomeric excess.

### EXAMPLE 4

A 600 ml stainless steel Parr reactor was charged with ethanol (170 ml), acetone (170 ml) and 6-chloro-3,5-dioxo-hexanoic acid tert-butyl ester (76 g). The reactor agitator was started and the speed set to 600 rpm. The reactor was pressurised using nitrogen to 7 bar and stirring continued for 5 minutes. After 5 minutes the reactor was slowly vented to ambient pressure, the pressurisation/depressurisation cycle was repeated for a total of five times to ensure complete removal of dissolved oxygen. At the end of the last cycle the reactor set-point temperature was adjusted to 95° C. (R)-[RuCl₂(BINAP)]n catalyst was accurately weighed (23 mg) into a catalyst transfer vessel and the vessel then purged using nitrogen for 5 minutes. The catalyst was flushed from the transfer vessel using deoxygenated solvent into a 100 ml stainless steel injection bomb which was attached to the Parr reactor. When the Parr reactor temperature was between 95° C. and 100° C. the injection bomb was pressurised to 100 bar using hydrogen. Appropriate valves were then opened to transfer the catalyst mixture and hydrogen into the reactor. The contents of the reactor were stirred at 600 rpm for 30 minutes before being cooled to less than 30° C. The reactor was then slowly vented to ambient pressure. The reactor contents were transferred into a 1 L rotary film evaporator flask and the mixture evaporated to constant weight by application of vacuum and by using a heated water bath. The residue was subjected to pot to pot distillation under vacuum to afford a clear colourless oily liquid product of 3R,5S-(6-chloromethyl-2,2-dimethyl-[1,3]dioxin-4-yl)-acetic acid tert-butyl ester in >95% yield, >95% purity and >98% enantiomeric excess.

### EXAMPLE 5

A feed tank was charged with 1.8 L acetone and 1.8 L methanol solvent. The solvent was deoxygenated by pumping it through a spray nozzle whilst pressurising to 7 bar with nitrogen and then depressurising through a needle valve at a controlled rate. The pressurisation/depressurisation cycle was repeated three times and the entire process automated using a PLC-based control system. In a similar manner a second feed tank was charged with ethyl-4-chloroacetoacetate (3.6 L) and deoxygenated using the same protocol to that described above. The catalyst, (R)-[RuCl₂(BINAP)]ₙ, (149 mg) was charged into a transfer vessel and the vessel purged using nitrogen before transferring the catalyst into the solvent feed tank. The catalyst solution had a concentration of 52.2 mg/Kg.

The two feed systems were connected to the continuous hydrogenation reactor system via two high-pressure pumps. The continuous hydrogenation reactor system was constructed of Hastalloy 276 and comprised a number of in-line static mixers to give a residence time of between 30 and 35 seconds. The static mixers also ensured good mixing of the process streams and rapid absorption of hydrogen. The reactor system was equipped with a recycle pump and an in-line valve which enabled operation as either a plug flow reactor (PFR, valve closed) or a continuous loop reactor (CLR, valve open). The system was equipped with a gas/liquid separator and the liquid level inside the separator controlled using a differential pressure sensor, which in turn operated an exit flow control valve. The reactor system was controlled using a PLC based control system. The hydrogenation reactor was pressurised using hydrogen and the pressure maintained between 90 and 100 bar by continually feeding hydrogen through a mass flow controller at a rate of 2.7 g/h. The reaction liquors passed through a heat exchanger using a pump such that the process temperature was maintained between 102° C. and 105° C.

The system above was operated as a plug flow reactor. The flow rate of the ethyl-4-chloroacetoacetate was set to 2.6 ml/minute and the flow rate of the catalyst solution set to 8.9 ml/min. These flows gave a process concentration of 30% w/w and a substrate to catalyst ratio of 20,000:1.

Over a series of continuous runs, each varying between 4 and 8 hours, the reactor consistently converted >99% ethyl-4-chloroacetoacetate to (S)-ethyl-4-chloro-3-hydroxybutyrate which was isolated after removing the solvents by evaporation to give a chemical yield of >98% and an enantiomeric excess greater than 99%.

### EXAMPLE 6

The reactor was set up as in Example 5, except it was operated as a continuous loop reactor. The flow rate of the ethyl-4-chloroacetoacetate was set to 2.55 ml/minute and the flow rate of the acetone/methanol catalyst solution set to 6.60 ml/min at a catalyst concentration of 45.8 mg/kg. These flows gave a process concentration of 37% w/w and a substrate to catalyst ratio of 65,000:1.

Over a series of continuous runs, each varying between 4 and 8 hours, the reactor consistently converted >99% ethyl-4-chloroacetoacetate to (S)-ethyl-4-chloro-3-hydroxybutyrate which was isolated after removing the solvents by evaporation to give a chemical yield of >98% and an enantiomeric excess greater than 99%.

What is claimed is:

1. A catalytic composition comprising a catalyst effective for catalysing asymmetric hydrogenation reactions, which catalyst requires acid activation, an acidic material effective for activating the catalyst, and a buffering compound or composition capable of forming, in the presence of the acidic material, an acetal, a ketal, a hemiacetal, and/or a hemiketal.

2. A catalytic composition according to claim 1, wherein the catalyst is a BINAP or other biarylbisphosphine-based ligand catalyst.

3. A catalytic composition according to claim 1, wherein the catalyst is effective for catalysing the enantioselective hydrogenation of p-ketoesters.

4. A catalytic composition according to claim 1, wherein the acidic material comprises a substrate suitable for asymmetric hydrogenation assisted by the catalyst.

5. A catalytic composition according to claim 4, wherein the substrate is ethyl-4-chloroacetoacetate.

6. A catalytic composition according to claim 1, wherein the buffering compound or composition comprises acetone and methanol.

7. A catalytic composition according to claim 1, wherein the buffering compound or composition is suitable for use as a solvent or solvent system in an asymmetric hydrogenation reaction carried out in the presence of the catalytic compostion.

8. A process for the enantioselective catalytic hydrogenation of a hydrogenatable substrate comprising contacting the substrate with hydrogen and with a catalyst effective for enantioselective hydrogenation of the substrate, which catalyst requires acid activation, in the presence of an acidic material and a buffering compound or composition capable of forming, in the presence of the acidic material, an acetal, a ketal, a hemiacetal, and/or a hemiketal, under conditions effective for enantioselective hydrogenation of the substrate.

9. A process according to claim 8, wherein the catalyst is a BINAP or otherbiarylbisphosphine-based ligand catalyst.

10. A process according to claim 9, wherein the catalyst is effective for catalysing the enantioselective hydrogenation of P-ketoesters.

11. A process according to claim 8, wherein the acidic material comprises a substrate suitable for asymmetric hydrogenation assisted by the catalyst.

12. A process according to claim 11, wherein the substrate is ethyl-4-chloroacetoacetate.

13. A process according to claim 8, wherein the buffering compound or composition comprises acetone and methanol.

14. A process according to claim 8, wherein the buffering compound or composition is suitable for use as a solvent or solvent system in the asymmetric hydrogenation reaction.

15. A process for the asymmetric catalytic hydrogenation of a substrate in the presence of an effective catalyst requiring acid activation, and of an acidic material for effecting such activation comprising the step of using a buffering compound or composition, which buffering compound or composition has the capacity to form an acetal, a ketal, a hemiacetal, and/or a hemiketal in the presence of the acidic material, to improve the enantiomeric excess of desired asymmetrically hydrogenated product.
